(19) United States
(12) Patent Application Publication
(10) Pub. No.: US 2014/0277087 A1
(43) Pub. Date: Sep. 18, 2014

(54) SPIRAL VENA CAVA FILTER REMOVAL DEVICE

(71) Applicant: COOK MEDICAL TECHNOLOGIES LLC, Bloomington, IN (US)

(72) Inventor: Samantha Manning, Taylor Mill, KY (US)

(73) Assignee: COOK MEDICAL TECHNOLOGIES LLC, Bloomington, IN (US)

(21) Appl. No.: 14/199,482

(22) Filed: Mar. 6, 2014

Related U.S. Application Data
(60) Provisional application No. 61/783,307, filed on Mar. 14, 2013.

(51) Int. Cl. A61B 17/50

(52) U.S. Cl. CPC A61B 17/50
USPC 606/200

(57) ABSTRACT

A retrieval device for removing filter devices from body cavities is provided. The retrieval device comprises at least an outer sheath, a first inner sheath, and a retrieval coil configured to coil around the outer surface of a filter device to initiate retraction. In a preferred embodiment, a second inner sheath is also provided. The two inner sheaths are configured to be slideable within the outer sheath. The second inner sheath contains a snaring wire. The snaring wire and the retrieval coil collaborate to dislodge a filter device from the vasculature, then the entire assembly is retracted and withdrawn. Particularly, the combined actions of the snaring wire and the retrieval coil facilitate the removal of filter devices that have become tilted or misaligned during deployment or treatment.

### CROSS-REFERENCE TO RELATED APPLICATIONS

This application claims the benefit of U.S. Provisional Application No. 61/783,307 filed on Mar. 14, 2013, entitled “SPIRAL TILTED VENA CAVA FILTER REMOVAL,” the entire contents of which are incorporated herein by reference.

### BACKGROUND

The present invention relates to medical devices. More particularly, the invention relates to an intravascular filter removal device that can be used to assist in the removal of a filter from the vena cava of a patient.

Filtering devices that are percutaneously placed in the vena cava have been available for a number of years. A need for such filtering devices arises in trauma patients, orthopedic surgery patients, neurosurgery patients, or in patients having medical conditions requiring bed rest or non-movement. Patients having such medical conditions face an increased risk of thrombosis in the peripheral vasculature, wherein thrombi break away from the vessel wall, risking downstream embolism or embolization. For example, depending on the size, such thrombi pose a serious risk of pulmonary embolism wherein blood clots migrate from the peripheral vasculature through the heart and into the lungs.

Historically, vena cava filters were considered to be permanent implants and remained implanted in the patient for life. More recently, removable vena cava filters have been developed. These filters may be removed from the patient's vena cava after the condition or medical problem that required the device has passed.

A particular problem that can arise during deployment of the filter or during treatment is misalignment of the filter. Many removable vena cava filters are manufactured with a removal hook at their proximal end. A physician or surgeon removing the filter can use a 2D imaging system to guide a looped snare to this hook, snare the hook, dislodge the filter from the vessel wall, and remove the filter from the patient's body.

Removal can be hindered or rendered impossible by minimally invasive means if the filter has become tilted during treatment. A practitioner may not be able to snare the hook of a tilted filter because some portion of the hook may have made contact with the vessel wall, and in some cases the vessel may have grown around it. In this case, methods that are less precise and more invasive than simple snaring, such as dislodging the filter with off label use of forceps, may have to be employed.

Development of a device with the purpose of removing a tilted vena cava filter has proven difficult.

### SUMMARY OF INVENTION

The present invention generally provides an intravascular filter removal device suitable for retrieval of a filter from a patient's vena cava, likely via the patient's jugular vein.

While the present invention has been described in terms of certain preferred embodiments, it will be understood that the invention is not limited to the disclosed embodiments, as those having skill in the art may make various modifications without departing from the scope of the following claims. Particular emphasis has been placed on solving the problem of removing a tilted or otherwise misaligned but it will be appreciated that the invention disclosed herein would also be capable of removing a properly-aligned vena cava filter.

In one embodiment, a removal device for an intravascular filter having an outer sheath with a proximal and a distal end is provided, comprising a first inner sheath extending from the proximal end to the distal end and having an interior lumen that extends to an opening at the distal end, the first inner sheath being disposed in the interior lumen of the outer sheath, and a retrieval coil disposed in the interior lumen of the first inner sheath, the retrieval coil being configured to grasp the intravascular filter. The removal device further comprises a second inner sheath extending from the proximal end to the distal end, the second inner sheath having an interior lumen that extends to an opening at the distal end. The first inner sheath and the second inner sheath are configured to slide independently of one another within the interior lumen of the outer sheath. The removal device further comprises a snare disposed in the interior lumen of the second inner sheath. The retrieval coil can be made of Nitinol and comprise a loop, and the snare can comprise a hook. Alternatively, the retrieval coil can be magnetic.

In another embodiment, the present invention provides a method of removing an intravascular filter from a vessel of a patient, the method comprising. In a first step, the outer sheath is inserted into the vessel. In a second step, the outer sheath is positioned over the inner sheath containing a retrieval coil so that the distal opening of the inner sheath is configured to accept the intravascular filter device to be removed. In a third step, the retrieval coil is extended through the distal opening of the inner sheath. In a fourth step, the retrieval coil is securely coiled around an exterior surface of the intravascular filter device. In a fifth step, the retrieval coil and its sheath are retracted together into the outer sheath until the intravascular filter device is positioned such that its hook is free of the vessel wall. In a sixth step, the second lumen is lowered through the lumen, positioned such that the snare is deployed to attach to for instance the hook of the filter. In a seventh step, then outer sheath is lowered over the both sheaths and the filter; the entire apparatus is withdrawn from the body of the patient.

Further objects, features, and advantages of the present invention will become apparent from consideration of the following description and the appended claims when taken in connection with the accompanying drawings.

### BRIEF DESCRIPTION OF THE DRAWINGS

The drawings are purely schematic illustrations of various aspects of the invention and are not necessarily to scale, unless expressly stated.

FIG. 1 is a perspective view of an intravascular filter removal device in accordance with the principles of the present invention.

FIGS. 2a-2e are cross-sectional views of the intravascular filter removal device illustrating various steps in the removal of an intravascular filter engaged within the vasculature of a patient.

### DETAILED DESCRIPTION

Provided herein is a device for removal of an intravascular filtration device that has been deployed in the vasculature of a patient. The filtration device is generally implanted in order to capture blood clots that may have formed so that they do not travel into portions of the body where their presence may cause damage. Most blood clots form in the lower limbs and are especially problematic if they reach the heart or lungs. As such, many filters are placed in the inferior vena cava, as this vein returns blood from the lower extremities to the heart. However, the device as described can also be used to extract filters from other veins as well.

The type of filters envisioned for removal by this device are roughly cone- or tulip-shaped filters which comprise a plurality of struts that diverge from an apical hub and are configured to engage the vessel wall at their opposite ends. These ends may terminate in hooks or barbs to more firmly engage the vessel wall. The filters are generally collapsible and are deployed percutaneously via catheter.

In some instances, a filter is intended to be implanted permanently; in others, the implantation may be temporary. In the latter case, the filter must be removed from the patient after the danger of embolism has passed. To this end, the filter is usually equipped with a retrieval hook at its apical hub. Using 2D imaging technology, a medical professional can guide retrieval catheter and a snaring wire through the vasculature. The snaring wire usually terminates in a hook of its own, which is used to engage the hook of the filter. The practitioner then frees the filter from the vessel wall and retracts the snaring wire into the retrieval catheter, the filter itself collapsing into the catheter as it is removed.

Retrieval can sometimes be more difficult if the intravascular filter has tilted during the deployment process or over the course of a period of treatment. In these cases, the hook of the filter can be difficult to access at best or can even have made sufficient contact to have become embedded into the vessel wall, with growth of new tissue around the hook making access impossible. Furthermore, a simple snare with a hook can cause damage if it engages with a part of the filter other than the hook. For instance, if the hook engages with one of the support struts of the filter, there is a possibility that the filter may become even more tilted or that the strut will become damaged by bending or kinking, which may in turn prevent closure or collapse of the filter as removal is attempted.

Due to the danger of bending, kinking, and breakage of the intravascular filter, care must be taken when removing a tilted or misaligned filter. The retrieval hook is the only portion of many of these filters that is designed to keep its structure when a force is applied; it is reinforced such that it can tolerate pushing and prying by the snaring hook, whereas the remainder of the device (that is, the filter body comprised of flexible struts) must be able to expand and collapse for the deployment and retrieval steps. Therefore, rather than risk the damage to the filter by applying excessive force to improper portions of the device, the best solution would be to manipulate the filter in a way that would avoid compromising its structure while regaining access to the retrieval hook.

The device described below can be used to remove a vena cava filter that resides in the vena cava in tilted fashion. One skilled in the art will recognize the advantage of using such a device to remove a tilted vena cava filter but will also note that the removal procedure can be used, with little to no modification, to remove a properly-aligned filter.

As used herein, the term “proximal” means “toward the medical practitioner” and “distal” means “away from the medical practitioner.” As a consequence, a filter deployed into the vena cava has its apical hub at its proximal end and its opening at its distal end, and blood flows through the filter from its distal to its proximal end.

The terms “substantially” or “about” used herein with reference to a quantity or shape include variations in the recited quantity or shape that are equivalent to the quantity or shape recited, such as an amount that is equivalent to the quantity recited for an intended purpose or function.

FIG. 1 illustrates a multicatheter intravascular retrieval device 10 for removing a tilted vena cava filter. The retrieval device 10 comprises an outer sheath 12. In this embodiment, a first inner sheath 20 and a second inner sheath 30 are disposed within an interior lumen 18 of the outer sheath 12. The proximal and distal ends of the outer sheath 12 define a longitudinal axis L.

In a typical procedure, the outer sheath 12 will be inserted percutaneously into the patient's jugular vein as a first step. To be a suitable fit for the jugular vein, the outer sheath will have a diameter of between about 5 and 15 French, more preferably between about 9 and 12 French, most preferably about 11.5 French. The outer sheath may be made of any suitable biocompatible material and may optionally be coated in a material that will increase biocompatibility, increase lubricity, or decrease trauma to the patient. The outer sheath has an interior lumen 18 that will contain the one or more inner sheaths and provide sufficient space for these to move independent of one another as necessary. As described below, the outer sheath will ultimately become the collapse point of the filter as it is drawn up into the interior lumen 18 during the final steps of the removal procedure and secures it during withdrawal.

The first inner sheath 20 has a proximal end and a distal end and is housed within the interior lumen 18 of outer sheath 12. As a result, first inner sheath 20 must have a smaller outer diameter than outer sheath 12 has an inner diameter. First inner sheath 20 is configured to move independently of outer sheath 12 as directed by the practitioner. Its primary purpose is to house and introduce retrieval coil 40. First inner sheath 20 may optionally be angled at its distal end.

The retrieval coil 40 is a flexible wire housed within the interior lumen 28 of first inner sheath 20. The retrieval coil 40 has relatively high elasticity and flexibility so that it can engage with filters of varying sizes and which are disposed in the vessel at various angles. In one embodiment, the retrieval coil is made of a memory metal. In a preferred embodiment, the memory metal is nitinol.

Medical devices having nitinol components are in widespread use. The advantage of this material is the ability to set a memorized shape at a particular transition temperature so that, when heated or cooled, a change in shape occurs. In one aspect of this invention, this transition temperature may be set to about body temperature and the shape change of the retrieval coil 40 may be from a relatively straight wire at room temperature to a spiral configuration with a series of circular turns that increase in diameter from the proximal to the distal end after insertion into the body. In another embodiment, the circular turns may be of essentially the same diameter, and in yet another embodiment, they may decrease in diameter from the proximal to the distal end. Embodiments wherein the turns of the retrieval coil 40 in its remembered configuration are not circular are envisioned, as are embodiments wherein there is only one loop or partial loop at the distal end.

The second inner sheath 30 has a proximal and a distal end and is disposed within the interior lumen 18 of the outer sheath 12. The diameter of the second inner sheath 30 may be equal to that of the first inner sheath 20, or it may be greater than or less than the diameter of the first inner sheath 20. However, if a second inner sheath 30 is included in the device, the diameters of both inner sheaths combined will be less than the inner diameter of the outer sheath 12 so that the two inner sheaths 20 and 30 will fit inside the interior lumen 18 and be capable of sliding parallel to the longitudinal axis L independent of one another. The distal end of second inner sheath 30 may optional bend inward toward the center of the vessel in order to bias the snaring wire 50 toward the probable location of the retrieval hook of the vena cava filter.

In another embodiment, the second inner sheath 30 is not included. In such an embodiment, the extraction of the filter is achieved solely by grasping the filter with the retrieval coil 40 and withdrawing the retrieval coil 40 and the first inner sheath 20 into the interior lumen 18 of outer sheath 12.

The second inner sheath 30, if present, houses snaring wire 50. Snaring a vena cava filter for removal is well known in the art and snaring wire 50 will serve this purpose in this invention. The snaring wire 50 can be made from a variety of materials including stainless steel, nitinol, INCONEL, a variety of plastics, or any other suitable material. In a preferred embodiment the snaring wire 50 should have limited flexibility as it should be capable of being retracted straight into the outer sheath 12 once engaged with the retrieval hook of the vena cava filter. However, certain applications may allow for more flexibility than others.

The distal end 52 of the snaring wire 50 is the portion that engages the retrieval hook of the vena cava filter. This distal end 52 can be configured in a number of ways to maximize the chances of successful retrieval. In a preferred embodiment, the distal end 52 comprises three hooks radially disposed at the end of the snaring wire at equal 120 degree increments around center. In this embodiment, the hooks are blunted at the end in order to minimize trauma to the vessel when it is deployed from the distal opening 38 of second inner sheath 30. This embodiment is advantageous because it provides three hooks that can potentially snare the filter and the hooks are spaced sufficiently far apart that they are able to freely engage the retrieval hook of the vena cava filter. The fact that the filter may not be in a predictable or easily accessible position increases the need for versatility in the configuration of the distal end 52 of the snaring wire 50.

Other configurations of the distal end 52 of the snaring wire 50 are envisioned as well. Instead of three hooks, the distal end 52 may comprise a single hook as in widely used retrieval devices, or there may be two, four, five, six, or more hooks at the distal end 52. In another embodiment, the distal end 52 may instead comprise a closed loop that fits around the retrieval hook of the vena cava filter. In yet another embodiment, the distal end 52 may comprise a magnet of any shape, provided that the retrieval hook of the vena cava filter is made of a magnetic material. The distal end 52 may comprise other configurations not explicitly listed here but which are capable of engaging a retrieval hook of a vena cava filter.

Turning now to FIG. 2a-2e, a multi-step process for engaging and retrieving a tilted vena cava filter is illustrated. The process illustrated encompasses the steps following percutaneous introduction of the outer sheath into the body vessel and concluding with the step preceding withdrawal of the device and the filter from the patient's body. This sequence is not to be considered exclusive or exhaustive as other intermediate steps may be included by practitioners.

FIG. 2a shows retrieval device 10 deployed within a body vessel 60 wherein a filter 70 that has become tilted is deployed. In this case, the retrieval hook 72 of the filter has been rendered inaccessible as it is embedded in vessel wall 62. The first inner sheath 20 has been moved out of outer sheath 12 and distal to filter 70.

Filter 70 as illustrated has a plurality of struts 76 that converge into a neck portion 74 and terminates in a retrieval hook 72. This is only an illustration of a single possible filter that can be retrieved by the retrieval device 10, however, and is illustrated this way for example's sake. Variations of the filter 70 include filters in which the struts 76 remain separate until meeting within an apical hub positioned between the struts 76 and the retrieval hook 72 and filters in which the retrieval apparatus is a shape other than a hook. Especially envisioned for retrieval of this device are the Cook Celect Vena Cava Filter and the Gunther Tulip Vena Cava Filter, although many different types of filters can be retrieved by this device.

In FIG. 2b, the retrieval coil has been partially extruded from first inner sheath 20 and is beginning to wrap around the outer surface of filter 70. The elasticity and flexibility of retrieval coil 40 allow it to wrap around any portion of the filter 70 that is accessible within vessel 60, optimally the neck portion 74 or around all of the struts 76. Care must be taken to surround all of the struts 76 with retrieval coil 40 as wrapping around only a few (or leaving only a few free of the coil) risks breaking or bending the filter, complicating the extraction process.

Once the practitioner is satisfied with the placement of the retrieval coil 40, the practitioner can maneuver the device 10 in a fitting manner in order to reposition filter 70 for retrieval. In FIG. 2c, the retrieval loop remains around the struts 76 of the filter 70 and the retrieval hook 72 has been dislodged from vessel wall 62.

FIG. 2d illustrates the next step in the retrieval process. Here the second inner sheath 30 has been deployed through the distal opening 14 of outer sheath 12 into body vessel 60. In the illustrated embodiment, the distal end of second inner sheath 30 is shown with an optional bend, the distal end angling toward the center of vessel 60. Snaring wire 50 is shown after being pushed out of second inner sheath 30 and its distal end 52 is engaged with retrieval hook 72. The snare can be advanced out of the inner sheath 30 by any means, including methods well known in the art such as loosening a Y-fitting of the external catheter and rotating a pin vice to maneuver the snaring wire 50 through the vessel.

FIG. 2e shows the filter 70, still engaged with the retrieval coil 40 and the distal end 52 of the snaring wire 50, being positioned for removal. Continued engagement with the retrieval coil 40 is optional if the practitioner judges that the filter 70 can be effectively retrieved without its assistance. As indicated by the arrows in FIG. 2e, the final step for removal comprises either moving the outer sheath 12 distally or withdrawing the first inner sheath 20 and the second inner sheath 30 into the interior lumen 18 of the outer sheath 12 in order to return the filter 70 to its contracted configuration. The entire retrieval device 10 can then be removed from the patient.

While the present invention has been described in terms of certain preferred embodiments, it will be understood that the invention is not limited to the disclosed embodiments, as those having skill in the art may make various modifications without departing from the scope of the following claims.

What is claimed is:

1. A removal device for an intravascular filter, the removal device comprising:
an outer sheath extending from a first proximal end to a first distal end defining a first longitudinal axis, the outer sheath having a first interior lumen and extending to a first opening at the first distal end;
a first inner sheath extending from a second proximal end to a second distal end defining a second longitudinal axis, the first inner sheath having a second interior lumen and extending to a second opening at the second distal end, the first inner sheath being disposed in the first interior lumen of the outer sheath;
a retrieval coil disposed in the second interior lumen of the first inner sheath, the retrieval coil being configured to be advanced distally along the second longitudinal axis and through the second opening of the first inner sheath, the retrieval coil being configured to grasp the intravascular filter and remove from the vessel wall a portion of the intravascular filter that is embedded in the vessel wall;
a second inner sheath extending from a third proximal end to a third distal end defining a third longitudinal axis, the second inner sheath having a third interior lumen and extending to a third opening at the third distal end, the second inner sheath being disposed in the first interior lumen of the outer sheath; and
a snaring wire disposed in the third interior lumen of the second inner sheath.

2. The removal device of claim 1 wherein each of the first inner sheath and the second inner sheath is configured to move longitudinally and along its respective longitudinal axis, independent of each other, within the first interior lumen of the outer sheath.

3. The removal device of claim 1 wherein the snaring wire has a free distal end, the snaring wire being configured to move distally along the third longitudinal axis and through the third opening of the second inner sheath, the free distal end configured to grasp the intravascular filter and retrieve the intravascular filter into the outer sheath while the retrieval coil is grasping the intravascular filter.

4. The removal device of claim 3 wherein the free distal end of the snaring wire comprises a hook.

5. The removal device of claim 3 wherein the free distal end of the snaring wire comprises a loop.

6. The removal device of claim 1 wherein the retrieval coil is made of Nitinol.

7. The removal device of claim 1 wherein the retrieval coil is magnetic.

8. The removal device of claim 1 wherein the retrieval coil being configured to grasp the intravascular filter comprises the retrieval coil being configured to coil around the intravascular filter as the retrieval coil is advanced distally through the second opening of the first inner sheath.

9. A method of removing an intravascular filter that is embedded in a vessel wall of a body vessel, the method comprising:
providing an outer sheath, the outer sheath extending from a first proximal end to a first distal end defining a first longitudinal axis, the outer sheath having a first interior lumen and extending to a first opening at the first distal end;
providing a first inner sheath, the first inner sheath extending from a second proximal end to a second distal end defining a second longitudinal axis, the first inner sheath having a second interior lumen and extending to a second opening at the second distal end, the first inner sheath being disposed in the first interior lumen of the outer sheath;
providing a retrieval coil, the retrieval coil disposed in the second interior lumen of the first inner sheath;
providing a second inner sheath extending from a third proximal end to a third distal end defining a third longitudinal axis, the second inner sheath having a third interior lumen and extending to a third opening at the third distal end, the second inner sheath being disposed in the first interior lumen of the outer sheath; and
providing a snaring wire having a free distal end, the snaring wire disposed in the third interior lumen of the second inner sheath;
percutaneously introducing the outer sheath into the body vessel;
advancing the first inner sheath distally along the first longitudinal axis and through the first opening of the outer sheath;
advancing the retrieval coil distally along the second longitudinal axis and through the second opening of the first inner sheath;
grasping the intravascular filter with the retrieval coil; and
retracting the retrieval coil proximally until a portion of the intravascular filter that is embedded in the vessel wall is removed from the vessel wall.

10. The method of claim 9 wherein the retrieval coil is made of Nitinol.

11. The method of claim 9 wherein the retrieval coil is magnetic.

12. The method of claim 9 further comprising:
advancing the second inner sheath distally along the first longitudinal axis and through the first opening of the outer sheath;
advancing the snaring wire distally along the third longitudinal axis and through the third opening of the second outer sheath;
grasping the intravascular filter with the free distal end while the retrieval coil is grasping the intravascular filter.

13. The method of claim 12 further comprising retrieving, by the snaring wire, the intravascular filter into the outer sheath while the retrieval coil is grasping the intravascular filter.

14. The method of claim 12 wherein the free distal end of the snaring wire comprises a hook.

15. The method of claim 12 wherein the free distal end of the snaring wire comprises a loop.

16. The method of claim 12 wherein grasping the intravascular filter with the retrieval coil comprises coiling the retrieval coil around the intravascular filter as the retrieval coil is advanced distally through the second opening of the first inner sheath.

17. A method of removing an intravascular filter that is embedded in a vessel wall of a body vessel, the method comprising:
providing a sheath extending from a proximal end to a distal end defining a longitudinal axis, the sheath having a lumen and extending to an opening at the distal end; and
providing a retrieval coil, the retrieval coil disposed in the sheath;
providing a snaring wire, the snaring wire disposed in the sheath;
percutaneously introducing the sheath into the body vessel;
advancing the retrieval coil distally along the longitudinal axis and through the opening of the sheath;
grasping the intravascular filter with the retrieval coil; and
retracting the retrieval coil proximally until the portion of the intravascular filter that is embedded in the vessel wall is removed from the vessel wall.
advancing the snaring wire distally along the longitudinal axis and through the opening of the sheath;
grasping the intravascular filter with the snaring wire while the retrieval coil is grasping the intravascular filter.

18. The method of claim 17 further comprising retrieving, by the snaring wire, the intravascular filter into the outer sheath while the retrieval coil is grasping the intravascular filter.

19. The method of claim 17 wherein grasping the intravascular filter with the retrieval coil comprises coiling the retrieval coil around the intravascular filter as the retrieval coil is advanced distally along the longitudinal axis.
